(19) United States
(12) Patent Application Publication
(10) Pub. No.: US 2006/0099573 A1
(43) Pub. Date: May 11, 2006

(54) Diagnostic assays

(76) Inventor: Maria-Jose Miguez, Miami Beach, FL (US)

Correspondence Address: YI LI, CUSPA TECHNOLOGY LAW ASSOCIATES, 11820 SW 107 AVENUE, MIAMI, FL, 33176, US

(21) Appl. No.: 11/087,645

(22) Filed: Mar. 24, 2005

Related U.S. Application Data
(60) Provisional application No. 60/555,713, filed on Mar. 24, 2004.
(60) Provisional application No. 60/602,033, filed on Aug. 17, 2004.

(51) Int. Cl. C12Q 1/70; G01N 33/53

(52) U.S. Cl. 435/5; 435/7.1

(57) ABSTRACT

Diagnostic assays using IgE for detecting HIV in a baby patient or a child patient that might be infected with HIV; distinguishing between classic dengue and dengue hemorrhagic fever in a patient that is believed to have either the classic or hemorrhagic version of dengue; and detecting cognitive impairment in a patient who is more than 40 years old.

### CROSS REFERENCE TO RELATED APPLICATIONS

This application claims priority to U.S. Provisional Patent Application Ser. No. 60/555,713, filed Mar. 24, 2004, and U.S. Provisional Patent Application Ser. No. 60/602,033, filed Aug. 17, 2004. U.S. Provisional Patent Application Ser. No. 60/555,713, filed Mar. 24, 2004, and U.S. Provisional Patent Application Ser. No. 60/602,033, filed Aug. 17, 2004, are herein incorporated by reference in their entirety.

### STATEMENT REGARDING FEDERALLY SPONSORED RESEARCH OR DEVELOPMENT

The name of the U.S. Government agency and the Government contract number are: 2D43TW00017 (Fogarty, now thought to be part of NIH).

### FIELD OF THE INVENTION

The present invention relates to methods for diagnosing diseases and to diagnostic assays, and particularly to a method for diagnosing infectious diseases using IgE antibodies instead of conventional IgG or IgM methods and also particularly to assays for diagnosing diseases based on IgE antibodies detection.

### SUMMARY OF THE INVENTION

Current advance have revealed that IgE, the most powerful amplifier of the immune response, may play a critical role in infectious diseases aside from its well-known role in allergy and parasites. Altered levels of total and specific IgE have been demonstrated in diseases of global importance including HHV, Pneumocystis Carinii, HIV, tuberculosis, dengue, and malaria. Although most researchers have attributed IgE elevations to polychonal and unspecific activation my research and data from others have demonstrated that IgE elevations are specifically directed to infectious pathogen antigens. The ability of IgE to employ a potent inflammatory reaction against pathogen is extremely effective. Moreover, IgE-dependent antigen capture and presentation is between 100 and 1000 fold more efficient than other antibodies in the immune response. In contrast to isotypes restricted to only one compartment, IgE has been described in vascular, mucosal, and tissue sections. Thus, we anticipate that IgE will have an important role in several infectious diseases.

The present invention is a method for diagnosis infectious diseases using IgE antibodies instead of conventional IgG or IgM methods and a diagnostic assay for diseases based on assaying for IgE antibodies. It is hypothesized that assaying for IgE antibodies will aid in detection of Alzheimer's disease, multiple sclerosis, Parkinson's disease, Down syndrome, and severe trauma of the central or peripheral neural system. These antibodies permit the early detection of a compromised CNS. Detection of elevated IgE antibodies that correlate with the aforementioned diseases, and severity thereof, can be used as a marker for the aforementioned diseases and aid medical physicians in the treatment thereof.

It is anticipated that elevations in IgE will be detected during malignancies and will be more severely increased as the disease progresses, as well as with the size of the tumor. It also hypothesized that IgE levels will rise with the appearance of metastasis. IgE levels will decline after successful treatment, and thus may be useful as both a prognostic marker and tool for treatment follow-up. Use of IgE as a diagnostic too may also exist for diseases caused by bioterrorism.

### DETAILED DESCRIPTION OF THE PREFERRED EMBODIMENTS

Human Immunodeficiency Viral Infection

A total of 170 serum samples were collected between 1987 and 1993 from HIV-1-infected (n=116) and HIV-1-seronegative (n=54) adults being monitored at the University of Miami School of Medicine. All samples were tested in the E. M. Papper Laboratory of Clinical Immunology by using duplicates, and the laboratory investigator was blinded as to the infection status. Blood specimens were collected, and serum or plasma samples were separated and stored at −20° F. until needed for the analyses. All samples were subjected to the following techniques: HIV PCR, ELISA, Western Blot using IgG, IgM and IgE.

Western Blot reactivity bands demonstrated that IgE reacted with the following HIV virus antigens: gp120, gp160 and gp 41. In addition, our work found that IgE strongly reacted with pol 66, gag 55, p32, env 18 and p24. While IgE response against gp120, 160 and 41 has been confirmed by other researchers and published (Maroone and Khalife), only our data indicate strong reactivity of IgE with pol 66, gag 55, p32, env 18 and p24. Analysis of the data demonstrated that IgE-based Western Blot for HIV antigens has high specificity (99%) and sensitivity (99%) with 95% confidence intervals. Thus, we are claiming that IgE reacts against these viral antigens, IgE based Western Blot is highly sensitive and specific.

In addition, 4 patients were initially considered reactive for IgE and were IgM and IgG seronegative by other serological techniques. Nonetheless, by PCR these patients were HIV positive. IgE early seroconversion reactivity was associated with positive IgE reactions to gp 160, gp120, pol 66, gag 55 and env 18. Thus, we claim that early detection of HIV is possible using IgE in patients that are in the acute phase and thus serologically negative by current commercially available techniques (ELISA/rapid test) but in actuality are HIV infected (PCR test). Last year in North Carolina, of 109,788 people tested, 622 were found to be HIV infected; of these, 21 were negative on the regular antibody test but found to be infected by directly testing for the virus.

Dengue Viral Infection

With regards to dengue, we have additional information that has not been published: IgE Dengue Diagnosis: If a cut-off level of >110 IU/ml, considered to be the highest marker of normal range, is selected, total IgE levels correctly identified primary dengue infection in 80% of the samples. Analysis of the data demonstrated a specificity (71%) and sensitivity (85%) using the IgE test, with 95% confidence intervals. Of interest, only one of the patients that had >200 IU/ml of IgE levels were not infected with dengue.

[image]

Acute Phase (n=3) Dengue DHF (n=5) A total of 8 samples were obtained from Ia Guajira, half of them corresponded to patients with dengue hemorrhagic fever. As shown in the Figure if a cut-off of >250 IU/ml is used we can diagnose dengue hemorrhagic fever with a 100% sensitivity and specificity of 71%. Therefore we are claiming that using IgE we can satisfactorily diagnoses classical dengue. In addition, using IgE we can distinguish classical dengue from dengue hemorrhagic fever.

Results of Dengue IgM Positives-IgE

| | | Dengue IgM | IgE IU/ML |
|---|---|---|---|
| Number | I.D. | ELISA | B.M. |
| 1 | 007 | Reactive | 346.54 |
| 2 | 044 | Reactive | 446.00 |
| 3 | 095 | Reactive | 446.00 |
| 4 | 097 | Reactive | 446.00 |
| 5 | 099 | Reactive | 147.03 |
| 6 | 107 | Reactive | 207.84 |
| 7 | 108 | Reactive | 446.00 |
| 8 | 114 | Reactive | 83.04 |
| 9 | 115 | Reactive | 136.37 |
| 10 | 116 | Reactive | 446.00 |
| 11 | 120 | Reactive | 27.62 |
| 12 | 122 | Reactive | 80.69 |
| 13 | 126 | Reactive | 76.73 |
| 14 | 128 | Reactive | 199.35 |
| 15 | 131 | Reactive | 446.00 |
| 16 | 132 | Reactive | 63.00 |
| 17 | 135 | Reactive | 446.00 |
| 18 | 140 | Reactive | 279.22 |
| 19 | 142 | Reactive | 256.20 |
| 20 | 144 | Reactive | 210.99 |
| 21 | 153 | Reactive | 446.00 |
| 22 | 159 | Reactive | 22.07 |
| 23 | 165 | Reactive | 446.00 |
| 24 | 169 | Reactive | 369.06 |
| 25 | 170 | Reactive | 446.00 |
| 26 | 173 | Reactive | 446.00 |
| 27 | 175 | Reactive | 205.09 |
| 28 | 178 | Reactive | 80.54 |
| 29 | 180 | Reactive | 446.00 |
| 30 | 182 | Reactive | 446.00 |
| 31 | 185 | Reactive | 446.00 |
| 32 | 186 | Reactive | 446.00 |
| 33 | 190 | Reactive | 446.00 |
| 34 | 193 | Reactive | 369.06 |
| 35 | 196 | Reactive | 347.74 |
| 36 | 205 | Reactive | 263.20 |
| 37 | 207 | Reactive | 446.00 |
| 38 | 208 | Reactive | 111.58 |
| 39 | 214 | Reactive | |
| 40 | 215 | Reactive | 282.55 |
| 41 | 216 | Reactive | 446.00 |
| 42 | 219 | Reactive | 92.76 |
| 43 | 223 | Reactive | 251.09 |
| 44 | 225 | Reactive | 433.45 |
| 45 | 231 | Reactive | 418.79 |
| 46 | 243 | Reactive | 446.00 |
| 47 | 244 | Reactive | 251.09 |
| 48 | 245 | Reactive | 117.86 |
| 49 | 254 | Reactive | 446.00 |
| 50 | 255 | Reactive | 446.00 |
| 51 | 257 | Reactive | 446.00 |
| 52 | 265 | Reactive | 446.00 |
| 53 | 267 | Reactive | 252.36 |
| 54 | 268 | Reactive | 168.04 |
| 55 | 271 | Reactive | 446.00 |
| 56 | 276 | Reactive | 159.49 |
| 57 | 280 | Reactive | 446.00 |

Normal range IgE: 1.50-100.00,
Unit: UI-ml

Results of Dengue IgM-IgE

| ID | Dengue IgM | IgE IU/ml | Mean |
|---|---|---|---|
| 1c | Reactive | 125.12 | 339 |
| 4c | Reactive | 446.00 | Mean Log 5.83 |
| 11c | Reactive | 446.00 | SD ± .516 |
| 13c | Reactive | 177.17 | |
| 14c | Reactive | 446.00 | |
| 17c | Reactive | 446.00 | |
| 29c | Reactive | 446.00 | |
| 31c | Reactive | 446.00 | |

Normal range IgE 0.00-100.00 UI/ml

IgE Western Blot by HIV Antigens

| | | | | | | | | | | | | ELISA | |
|---|---|---|---|---|---|---|---|---|---|---|---|---|---|
| | ELISA | Western | | | | | | | | | Blank | IgE | RES |
| NUMBER | IgG | Blot | 160 | 120 | 66 | 55 | 41 | 32 | 24 | 18 | 492/620 | 492/620 | IgE |
| 1 | SR | NR | NR | NR | NR | NR | NR | NR | NR | NR | 0.117 | 0.194 | NR |
| 69722 | SR | NR | SR | NR | NR | NR | NR | NR | NR | NR | 0.110 | 0.190 | NR |
| 69723 | SR | NR | SR | NR | NR | NR | NR | NR | NR | NR | 0.112 | 0.190 | NR |
| 69919 | NR | NR | NR | NR | NR | NR | NR | NR | NR | NR | 0.050 | 0.148 | NR |
| 70152 | SR | NR | NR | NR | NR | NR | NR | NR | NR | NR | 0.243 | 0.350 | IND |
| 70691 | NR | NR | NR | NR | NR | NR | NR | NR | NR | NR | 0.122 | 0.198 | NR |
| 71428 | SR | PR | SR | SR | WR | WR | WR | NR | SR | SR | 0.767 | 0.866 | PR |
| 72079 | | NR | NR | NR | NR | NR | NR | NR | NR | NR | 0.104 | 0.163 | NR |
| 72936 | WR | NR | NR | NR | NR | NR | NR | NR | NR | NR | 0.196 | 0.260 | NR |
| 72995 | WR | IND | WR | NR | NR | NR | NR | NR | NR | NR | 0.400 | 0.436 | IND |
| 73017 | NR | NR | NR | NR | NR | NR | NR | NR | NR | NR | 0.024 | 0.120 | NR |
| 73392 | MR | NR | NR | NR | NR | NR | NR | NR | NR | NR | 0.083 | 0.144 | NR |
| 74039 | NR | NR | NR | NR | NR | NR | NR | NR | WR | NR | 0.030 | 0.092 | NR |
| 75828 | SR | PR | SR | SR | SR | SR | SR | SR | SR | SR | 0.753 | 0.810 | PR |
| 75838 | WR | SR | WR | WR | WR | WR | NR | NR | NR | NR | 0.275 | 0.284 | IND |
| 75864 | MR | NR | NR | NR | NR | NR | NR | NR | NR | NR | 0.036 | 0.068 | NR |
| 75906 | SR | NR | NR | NR | NR | NR | NR | NR | NR | NR | 0.074 | 0.184 | NR |
| 75943 | SR | PR | SR | SR | SR | SR | SR | SR | SR | SR | 0.764 | 0.801 | PR |
| 75994 | SR | PR | SR | SR | SR | SR | SR | SR | SR | SR | 0.661 | 0.691 | PR |
| 76065 | SR | PR | SR | SR | SR | SR | SR | SR | SR | SR | 0.713 | 0.770 | PR |
| | | | | | | | | | | | | ELISA | |
| | ELISA | | | | | | | | | | Blank | IgE | RES |
| NUMBER | IgG | WB | 160 | 120 | 66 | 55 | 41 | 32 | 24 | 18 | 492/620 | 492/620 | IgE |
| 76094 | SR | PR | SR | SR | SR | SR | SR | SR | SR | SR | 0.686 | 0.730 | PR |
| 76096 | WR | NR | NR | NR | NR | NR | NR | NR | NR | NR | 0.089 | 0.120 | NR |
| 76190 | WR | NR | NR | NR | NR | NR | NR | NR | NR | NR | 0.060 | 0.234 | NR |
| 76844 | NR | NR | NR | NR | NR | NR | NR | NR | NR | NR | | contam | |
| 77136 | NR | NR | NR | NR | NR | NR | NR | NR | NR | NR | 0.018 | 0.096 | NR |
| 77670 | SR | PR | SR | SR | WR | WR | WR | NR | WR | NR | 1.125 | 1.165 | PR |
| 77740 | WR | NR | NR | NR | NR | NR | NR | NR | NR | NR | 0.007 | 0.090 | NR |
| 78692 | MR | NR | NR | NR | NR | NR | NR | NR | NR | NR | 0.012 | 0.070 | NR |
| 78884 | SR | NR | NR | NR | NR | NR | NR | NR | NR | NR | 0.040 | 0.107 | NR |
| 78986 | SR | PR | SR | SR | SR | WR | SR | SR | SR | SR | 0.650 | 0.758 | PR |
| 79137 | WR | NR | NR | NR | NR | NR | NR | NR | NR | NR | 0.066 | 0.135 | NR |
| 79156 | SR | PR | SR | SR | SR | SR | SR | SR | SR | SR | 0.708 | 0.778 | PR |
| 79317 | NR | NR | NR | NR | NR | NR | NR | NR | NR | NR | 0.030 | 0.085 | NR |
| 79763 | SR | PR | SR | SR | SR | SR | SR | SR | SR | SR | 0.752 | 0.800 | PR |
| 79790 | MR | NR | NR | NR | NR | NR | NR | NR | NR | NR | 0.024 | 0.096 | NR |
| 79892 | NR | NR | NR | NR | NR | NR | NR | NR | NR | NR | 0.045 | 0.119 | NR |
| 80026 | NR | NR | | | | | | | | | 0.094 | 0.127 | NR |
| 80035 | SR | PR | SR | SR | SR | SR | SR | SR | SR | SR | 0.571 | 0.643 | PR |
| 80039 | SR | PR | SR | SR | SR | SR | SR | SR | WR | SR | 0.794 | 0.866 | PR |
| 80140 | SR | PR | SR | SR | SR | WR | SR | SR | SR | WR | 0.596 | 0.647 | PR |
| 80185 | SR | PR | SR | SR | SR | WR | SR | SR | WR | SR | 0.680 | 0.734 | PR |
| 80300 | SR | PR | SR | SR | SR | SR | SR | SR | SR | SR | 0.893 | 0.957 | PR |
| 80344 | SR | PR | SR | SR | SR | SR | SR | SR | SR | SR | 0.680 | 0.726 | PR |
| 80356 | SR | PR | SR | SR | SR | SR | SR | SR | SR | SR | 0.670 | 0.748 | PR |
| 80682 | SR | PR | SR | SR | WR | WR | SR | WR | WR | SR | 0.634 | 0.633 | PR |
| 80794 | | NR | NR | NR | NR | NR | NR | NR | NR | NR | 0.040 | 0.073 | NR |
| 80968 | SR | PR | SR | SR | WR | WR | WR | WR | SR | SR | 0.751 | 0.805 | PR |
| 81071 | SR | PR | SR | SR | SR | WR | SR | SR | WR | NR | 0.650 | 0.631 | PR |
| 81072 | SR | PR | SR | SR | SR | WR | SR | WR | SR | WR | 0.643 | 0.648 | PR |
| 81135 | NR | NR | NR | NR | NR | NR | NR | NR | NR | NR | 0.023 | 0.054 | NR |
| 81141 | SR | PR | SR | SR | SR | WR | SR | SR | SR | WR | 0.698 | 0.706 | PR |
| 81212 | SR | PR | SR | SR | WR | WR | SR | NR | SR | WR | 0.577 | 0.569 | PR |
| 81271 | SR | PR | SR | SR | SR | WR | SR | SR | WR | NR | 0.656 | 0.652 | PR |
| 81232 | SR | IND | NR | NR | WR | WR | NR | NR | SR | NR | 0.270 | 0.282 | IND |
| 81 | PR | PR | SR | SR | SR | SR | SR | SR | SR | SR | 0.600 | 0.632 | PR |
| R | PR | PR | WR | WR | SR | SR | SR | SR | SR | SR | 0.873 | 0.915 | PR |
| 77 | PR | PR | SR | WR | SR | SR | SR | SR | SR | SR | 0.985 | 1.032 | PR |
| 65 | PR | PR | SR | WR | SR | SR | SR | SR | SR | SR | 0.904 | 0.956 | PR |
| 7445 | PR | PR | SR | SR | SR | SR | SR | SR | SR | SR | 0.765 | 0.797 | PR |
| 76 | PR | PR | SR | SR | SR | SR | SR | SR | SR | SR | 0.939 | 0.975 | PR |
| 0 | PR | PR | SR | SR | SR | SR | SR | SR | SR | SR | 0.670 | 0.735 | PR |
| 79 | PR | PR | SR | SR | WR | WR | SR | SR | SR | SR | 0.943 | 1.012 | PR |
| 71509 | NR | NR | | | | | | | | | 0.020 | 0.700 | NR |
| 71507 | NR | NR | | | | | | | | | 0.006 | 0.088 | NR |
| 75084 | PR | PR | SR | SR | SR | SR | SR | SR | SR | SR | 0.859 | 0.943 | PR |
| 74835 | PR | PR | SR | SR | SR | SR | SR | SR | SR | SR | 0.693 | 0.740 | PR |
| 79156 | PR | PR | SR | SR | SR | WR | WR | WR | WR | SR | 0.708 | 0.788 | PR |
| M2 | NR | NR | | | | | | | | | 0.032 | 0.080 | NR |
| | | | | | | | | | | | | ELISA | |
| | ELISA | | | | | | | | | | Blank | IgE | RES |
| Number | IgG | WB | 160 | 120 | 66 | 55 | 41 | 32 | 24 | 18 | 492/620 | 492/620 | IgE |
| 70605 | WP | PR | WR | WR | WR | NR | NR | SR | SR | SR | 0.950 | 1.262 | PR |
| 77436 | WP | PR | SR | SR | SR | SR | SR | SR | SR | SR | 0.680 | 0.733 | PR |
| 27 | SR | PR | SR | SR | SR | WR | SR | SR | SR | SR | 1.535 | 1.637 | PR |
| 39 | SR | PR | SR | SR | SR | SR | SR | SR | SR | SR | 1.559 | 1.656 | PR |
| 90 | SR | PR | SR | SR | SR | SR | SR | SR | SR | SR | 1.221 | 1.317 | PR |
| 19 | SR | PR | SR | SR | SR | SR | SR | SR | SR | SR | 1.188 | 1.278 | PR |
| 28 | SR | PR | SR | SR | SR | SR | SR | SR | SR | SR | 1.058 | 1.153 | PR |
| 3O | SR | PR | SR | SR | SR | SR | SR | SR | SR | SR | 1.038 | 1.136 | PR |
| 31 | SR | PR | WR | NR | SR | SR | SR | SR | SR | SR | 0.653 | 0.749 | PR |
| 201 | NR | NR | | | | | | | | | 0.160 | 0.255 | NR |
| 202 | NR | NR | | | | | | | | | 0.088 | 0.178 | NR |
| 203 | NR | NR | | | | | | | | | 0.093 | 0.183 | NR |
| 205 | NR | NR | | | | | | | | | 0.019 | 0.114 | NR |
| 207 | NR | NR | | | | | | | | | 0.018 | 0.108 | NR |
| 209 | NR | NR | | | | | | | | | 0.043 | 0.133 | NR |
| 220 | NR | NR | | | | | | | | | 0.007 | 0.097 | NR |
| 208 | NR | NR | | | | | | | | | 0.030 | 0.107 | NR |
| 214 | NR | NR | | | | | | | | | 0.020 | 0.110 | NR |
| 215 | NR | NR | | | | | | | | | 0.025 | 0.112 | NR |
| 217 | NR | NR | | | | | | | | | 0.038 | 0.128 | NR |
| 218 | NR | NR | | | | | | | | | 0.047 | 0.143 | NR |
| 220 | NR | NR | | | | | | | | | 0.071 | 0.163 | NR |
| 221 | NR | NR | | | | | | | | | 0.059 | 0.149 | NR |
| 225 | NR | NR | | | | | | | | | 0.080 | 0.172 | NR |
| 210 | NR | NR | | | | | | | | | 0.078 | 0.148 | NR |
| 212 | NR | NR | | | | | | | | | 0.044 | 0.134 | NR |
| 213 | NR | NR | | | | | | | | | 0.048 | 0.1137 | NR |
| 219 | NR | NR | | | | | | | | | 0.057 | 0.167 | NR |
| 226 | NR | NR | | | | | | | | | 0.036 | 0.102 | NR |
| 228 | NR | NR | | | | | | | | | 0.028 | 0.147 | NR |
| 230 | NR | NR | | | | | | | | | 0.028 | 0.147 | NR |
| 233 | NR | NR | | | | | | | | | 0.025 | 0.115 | NR |
| 235 | NR | NR | | | | | | | | | 0.025 | 0.115 | NR |
| 222 | NR | NR | | | | | | | | | 0.037 | 0.133 | NR |
| 224 | NR | NR | | | | | | | | | 0.030 | 0.124 | NR |
| 227 | NR | NR | | | | | | | | | 0.030 | 0.123 | NR |
| 229 | NR | NR | | | | | | | | | 0.040 | 0.136 | NR |
| 231 | NR | NR | | | | | | | | | 0.080 | 0.173 | NR |
| 234 | NR | NR | | | | | | | | | 0.028 | 0.119 | NR |
| 240 | NR | NR | | | | | | | | | 0.023 | 0.113 | NR |
| 258 | NR | NR | | | | | | | | | 0.020 | 0.112 | NR |
| 253 | NR | NR | | | | | | | | | 0.010 | ″0.098 | NR |
| 236 | NR | NR | | | | | | | | | 0.035 | 0.130 | NR |
| 247 | NR | NR | | | | | | | | | 0.038 | 0.139 | NR |
| 248 | NR | NR | | | | | | | | | 0.050 | 0.164 | NR |
| 249 | NR | NR | | | | | | | | | 0.052 | 0.166 | NR |
| 250 | NR | NR | | | | | | | | | 0.055 | 0.159 | NR |
| 1 | WR | WR | SR | SR | S | R | SR | SR | SR | SR | 0.017 | | NR |
| | | | | | | | | | | | | ELISA | |
| | ELISA | | | | | | | | | | Blank | IgE | RES |
| NUMBER | IgG | WB | 160 | 120 | 66 | 55 | 41 | 32 | 24 | 18 | 492/620 | 492/620 | IgE |
| 3O | SR | PR | SR | SR | SR | SR | SR | SR | SR | SR | 0.808 | 0.910 | PR |
| 44 | SR | PR | SR | NR | NR | SR | SR | SR | SR | SR | 0.811 | 0.925 | PR |
| 13 | SR | PR | SR | WR | WR | WR | SR | SR | SR | SR | 0.779 | 0.866 | PR |
| 38 | SR | PR | SR | SR | SR | SR | SR | SR | SR | SR | 0.768 | 0.865 | PR |
| 54 | SR | PR | SR | SR | SR | SR | SR | SR | SR | SR | 0.748 | 0.831 | PR |
| 55 | SR | PR | SR | SR | SR | SR | SR | WR | SR | SR | 0.783 | 0.902 | PR |
| 61 | SR | PR | SR | SR | SR | SR | SR | SR | SR | SR | 0.074 | 0.829 | PR |
| 16 | SR | PR | | | | | | | | | 0.755 | 0.853 | PR |
| 66 | SR | PR | SR | SR | SR | SR | SR | SR | SR | SR | 0.083 | 0.913 | PR |
| 76 | SR | PR | SR | SR | SR | SR | SR | SR | SR | SR | 0.778 | 0.870 | PR |
| 3 | SR | PR | SR | SR | SR | SR | SR | WR | SR | WR | 0.950 | 1.001 | PR |
| 4 | SR | PR | | | | | | | | | 1.001 | 1.010 | PR |
| 1 | SR | PR | WR | WR | SR | SR | SR | SR | SR | SR | 0.980 | 0.988 | PR |
| 93 | SR | PR | SR | SR | SR | SR | SR | SR | SR | SR | 1.020 | 1.038 | PR |
| 14 | SR | PR | SR | SR | SR | SR | SR | SR | SR | SR | 0.998 | 1.017 | PR |
| 38 | SR | PR | SR | SR | SR | SR | SR | SR | SR | SR | 0.921 | 0.911 | PR |
| 52 | SR | PR | | | | | | | | | 0.945 | 0.949 | PR |
| 53 | SR | PR | SR | WR | SR | SR | SR | SR | SR | SR | 0.914 | 0.950 | PR |
| 64 | SR | PR | SR | SR | SR | SR | SR | SR | SR | SR | 0.968 | 1.027 | PR |
| 55 | SR | PR | SR | SR | SR | SR | SR | WR | SR | SR | 1.000 | 1.022 | PR |
| 56 | SR | PR | | | | | | | | | 1.024 | 1.027 | PR |
| 57 | SR | PR | | | | | | | | | 1.019 | 1.025 | PR |
| 58 | SR | PR | SR | SR | SR | SR | SR | SR | SR | SR | 0.936 | 0.991 | PR |
| 59 | SR | PR | | | | | | | | | 0.836 | 0.935 | PR |
| 60 | SR | PR | SR | SR | SR | SR | SR | SR | SR | SR | 0.886 | 0.971 | PR |
| 61 | SR | PR | SR | SR | SR | SR | SR | SR | SR | SR | 0.884 | 0.960 | PR |
| 66 | SR | PR | SR | SR | SR | SR | SR | SR | SR | SR | 0.663 | 0.754 | PR |
| 86 | SR | PR | SR | SR | SR | SR | SR | SR | SR | SR | 0.917 | 0.998 | PR |
| 84 | SR | PR | SR | SR | SR | SR | SR | SR | SR | SR | 0.880 | 0.952 | PR |
| 62 | SR | PR | SR | SR | SR | SR | SR | SR | SR | SR | 0.984 | 1.021 | PR |
| 63 | SR | PR | WR | WR | SR | SR | SR | SR | SR | SR | 0.981 | 1.005 | PR |
| 8O | SR | PR | SR | SR | SR | SR | SR | SR | SR | SR | 0.946 | 0.968 | PR |
| 97 | SR | PR | SR | SR | SR | SR | SR | SR | SR | SR | 0.915 | 0.960 | PR |
| 76844 | NR | NR | NR | NR | NR | NR | NR | NR | NR | NR | NR | 0.241 | NR |
| 74835 | SR | PR | SR | SR | SR | SR | SR | SR | SR | SR | 0.818 | 0.920 | PR |
| 74836 | SR | PR | SR | SR | SR | SR | SR | SR | SR | SR | 0.878 | 0.883 | PR |
| 74837 | SR | PR | WR | WR | SR | SR | SR | SR | SR | SR | 0.991 | 1.000 | PR |
| 74838 | SR | PR | SR | SR | SR | SR | SR | SR | SR | WR | 0.977 | 0.991 | PR |
| 74839 | SR | PR | SR | SR | SR | SR | SR | SR | SR | SR | 0.923 | 0.937 | PR |
| 208 | NR | NR | | | | | | | | | 0.060 | 0.107 | NR |
| 81201 | WR | SR | SR | SR | SR | SR | SR | SR | SR | SR | 0.863 | 0.892 | PR |
| 81319 | SR | PR | SR | SR | SR | SR | SR | SR | SR | | 0.848 | 0.874 | PR |
| 81396 | WR | PR | SR | WR | SR | SR | SR | SR | SR | | 0.817 | 0.879 | PR |
| 81632 | SR | PR | SR | SR | SR | SR | SR | SR | SR | | 0.807 | 0.853 | PR |
| 235 | NR | NR | | | | | | | | | 0.091 | 0.141 | NR |
| 81135 | WR | NR | NR | NR | NR | NR | NR | NR | NR | | 0.103 | 0.156 | NR |
| 81621 | SR | PR | SR | SR | SR | SR | SR | SR | WR | SR | 0.838 | 0.865 | PR |
| 81399 | SR | PR | SR | SR | SR | SR | SR | WR | SR | WR | 0.883 | 0.899 | PR |

HIV Study

We are incorporating a summary table produced in 1993 by the administrator. We are also attaching one original page from the ELISA reader of patient numbers 39-214 dated June 1992 as a proof of the veracity of these results.
Column 1 Represents the laboratory number of the patient
Column 2 Indicates the result with the ELISA IgG (SR=positive, MR=positive WR=indeterminate and NR=negative)
Column 3 Indicates the final result using a conventional Western Blot (NR=negative SR=positive PR=Positive WR=represents an indeterminate result and for those cases a PCR was performed)
Columns 4-11 Represents the IgE Western Blot and shows in detail which HIV antigens (gp 160, gp120, gp66, pol66, gag55, gp41, tat32, nucleocapsid proteins 24 and 18) are bound to IgE.
Column 13 As samples for the ELISAS were run in duplicate, the numbers in column 13 represents the mean reading using filters 492/620.
Column 14 Illustrates the final IgE interpretation of the test as follows: if the reading in Column 13 was below 0.260 the IgE ELISA was considered negative (NR). It lecture was above 0.569 it was clearly positive (PR).

As shown in Table 1. All patients were subject to IgG ELISAS, a commercial Western Blot, an IgE Western Blot and IgE ELISA all for the detection of HIV. If the commercial WB was indeterminate, or the IgE was indeterminate, samples were submitted to viral culture/PCR to establish the accurate HIV status of the patient. As recommended by the CDC, all ELISAS were run in duplicate.

How to Read the Table:

Patient 70691 was negative according to the IgG ELISA (column #2-NR), and the conventional Western Blot (column #3-NR), non-reactive to each of the HIV antigens using IgE (columns 4-11) therefore, this patient was IgE negative in the Western Blot and negative according to the ELISA IgE (column 14). In summary, the 4 test (IgG/ELISA, IgG/Western Blot, IgE/ELISA, IgE/Western Blot) concord in the identification of HIV infection in patient 70691 was determined to be negative.

Patient #71428 was HIV positive according to the IgG ELISA (column #2-SR), and the commercial western Blot (column#3-PR) and IgE bound strongly to HIV antigens gp160 (column #4-SR), gp120 (#5-SR), gag 24 (#10-SR) and envelope 18 (#11-SR). Week bonds were indicated between IgE and HIV antigens pol 66 (#6-WR), gag 55(#7-WR), gp41 (#8-WR). Thus, according to the IgE Western Blot, this patient was determined to be HIV positive. By the IgE based ELISA (column #14) the patient was confirmed positive. In other words, the IgE ELISA and the IgE Western Blot were equally effective in diagnosed an HIV infected individual.

Finally, patient 75864 was positive according to the IgG ELISA (MR), but negative by conventional Western Blot (NR). IgE did not react with any of the HIV antigens, indicating IgE Western Blot negativity and subsequent negative results were obtained in the IgE ELISA. In this case, the IgE ELISA and the IgE Western Blot correctly identified the patient as HIV negative, whereas the IgG ELISA resulted in a false positive.

Four indeterminate (IND) IgE cases occurred, and all but one patient (70152) was HIV infected at the acute phase of seroconversion (the appearance of specific antibodies, usually occurs 3 to 12 weeks after immune-recognition of the virus antigens).

Analysis of the data demonstrated a high specificity (96%) and sensitivity (98%) of the Western Blot IgE test, with 95% confidence intervals. The ELISA IgE demonstrated a high specificity (99%) and sensitivity (99%) of the IgE test, with 95% confidence intervals.
1 Thus we are claiming that serological test using IgE are ABLE to diagnose the presence or not of HIV infection.
2 We claim that IgE diagnostic tests are able to diagnose HIV infection even at early acute stages of seroconversion.

Data also indicate that specific IgE is generated against the full spectrum of HIV antigens. Our data indicate for the first time that IgE binds gp160, gp120, pol 66, gag 55, gp 41, gp 32, gp24 and envelope 18
1 We claim that IgE binds to gp 41, gp 32, pol 66, gag55 and env 18
2 We claim that these antibody-antigen complex are useful for the diagnosis of HIV infection in both children and adults.
   Dengue

Please note that we are sending a copy of the laboratory results (1996). The first two pages are the results of samples obtained in San Andres, where only Classic Dengue occurred. Four columns exist in the report.

| | |
|---|---|
| Column 1 | Number |
| Column 2 | Laboratory ID |
| Column 3 | Dengue Status |
| Column 4 | IgE levels |

IgE Dengue Diagnosis: If a cut-off level of >110 IU/ml, considered to be the highest marker of normal range, is selected, total IgE levels correctly identified primary dengue infection in 80% of the samples. Analysis of the data demonstrated a specificity (71%) and sensitivity (85%) using the IgE test, with 95% confidence intervals. Of interest, only one of the patients that had >200 IU/ml of IgE levels was not infected with dengue. Previous studies, including our own work, have described elevated total and specific IgE levels associated with dengue, but this is the first time that IgE's potential as a diagnostic tool has been claimed.

We are claiming that IgE is useful in diagnosing dengue infection. The second page represents 8 cases of dengue hemorrhagic fever that occurred in Ia Guajira. For the analyses, IgE from patients with dengue hemorrhagic fever (La Guajira) were compared with IgE from patients infected with classic dengue (San Andres). Please see that statistically significant differences are reached (see attached page). Findings indicated that IgE has a sensitivity of 75% in diagnosing dengue hemorrhagic fever, and these results were statistically significant (p=0.003).

| | |
|---|---|
| Column 1 | Laboratory ID |
| Column 2 | Dengue Status as per IgM |
| Column 3 | IgE levels. |

| | Dengue Hemorrhagic | Classic Dengue |
|---|---|---|
| >450 IU/ml | 6 | 17 |
| <450 IU/ml | 2 | 74 |

Sensitivity 75%

We are claiming that IgE can distinguish classic dengue from dengue hemorrhagic fever.

IgE for the Diagnosis of Cognitive Impairment

1. IgE levels were higher in the patients older than 40 years (387±331 IU/ml) relative to Younger patients (329±341 IU/ml). Lower IgE levels were found in HIV(−) patient less than 45 years of age (n=11; 97±145), compared to older seronegatives>45 (n=3; 198±246 IU/ml).

2. Mean IgE levels were significantly lower in patients with normal MMSE total scores (>28) 238±326 IU/ml, compared to those who had moderate cognitive impairment (scored <27) 483±336, p=0.04. Measurements of IgE levels were even higher in the severely impaired group (<24): 73% had more than 200 IU/ml of total IgE. Univariate analyses demonstrated that patients with IgE>200 IU/ml were 6 times more likely to be impaired (MMSE score <24), as compared to those with <200 IgE IU/ml (p=0.02).

Prognostic Value of Total IgE in Mental Function: Univariate analyses indicated that patients with IgE >200 at baseline were 3 times more likely to be cognitively impaired (score<24, P=0.01) at the 2 year visit.

In summary, these preliminary findings indicate that elevated IgE levels are 1) evident in older adults 2) associated with mental impairment 3) may be sensitive prognostic marker of cognitive impairment. Thus we are claiming that IgE could be used as a diagnostic tool for cognitive impairment. I am including laboratory report to demonstrate the veracity of the analyzed data.

| | 1 | 2 | 3 | 4 | 5 | 6 | 7 | 8 | 9 | 10 | 11 | 12 |
|---|---|---|---|---|---|---|---|---|---|---|---|---|
| A | | +39 | +66 | −205 | | | | | | | | |
| | 2.590 | 0.153 | 0.703 | 0.085 | 0.001 | 0.001 | 0.001 | 0.001 | 0.001 | 0.001 | 0.001 | 0.001 |
| B | | +80 | +64 | −206 | | | | | | | | |
| | 2.600 | 0.147 | 0.653 | 0.076 | 0.000 | 0.000 | 0.000 | 0.000 | 0.000 | 0.000 | 0.000 | 0.000 |
| C | | +90 | +65 | −207 | | | | | | | | |
| | OVERFLOW | OVERFLOW | 2.042 | 0.070 | 0.000 | 0.000 | 0.000 | 0.000 | 0.000 | 0.000 | 0.000 | 0.000 |
| D | | +56 | +61 | −208 | | | | | | | | |
| | OVERFLOW | OVERFLOW | 2.145 | 0.071 | 0.000 | 0.000 | 0.000 | 0.000 | 0.000 | 0.000 | 0.000 | 0.000 |
| E | | +54 | −201 | −209 | | | | | | | | |
| | 0.092 | 2.159 | 0.092 | 0.071 | 0.000 | 0.000 | 0.000 | 0.000 | 0.000 | 0.000 | 0.000 | 0.000 |
| F | | +59 | −202 | −210 | | | | | | | | |
| | 0.114 | 2.252 | 0.072 | 0.069 | 0.000 | 0.000 | 0.000 | 0.000 | 0.000 | 0.000 | 0.000 | 0.000 |
| G | +37 | −99 | −203 | −212 | | | | | | | | |
| | 0.861 | 0.074 | 0.079 | 0.074 | 0.000 | 0.000 | 0.000 | 0.000 | 0.000 | 0.000 | 0.000 | 0.000 |
| H | +38 | −81 | −204 | −214 | | | | | | | | |
| | 0.000 | 0.095 | 0.109 | 0.074 | 0.000 | 0.000 | 0.000 | 0.000 | 0.000 | 0.000 | 0.000 | 0.000 |

Statistic Analysis

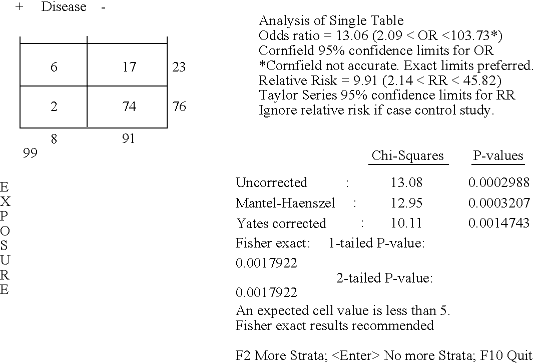

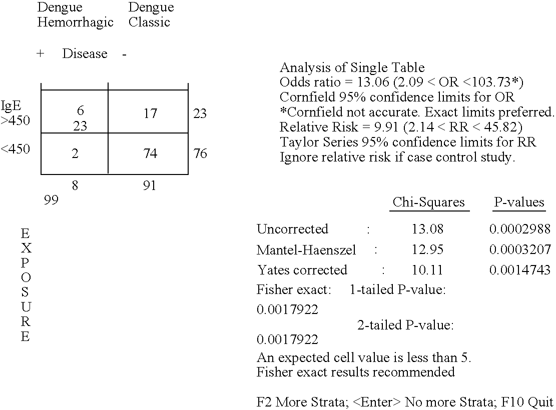

| | | ASSIGNED | | Calc | CV | % |
|---|---|---|---|---|---|---|
| NAME | WELL | IU/ml | O.D. | IU/ml | % | Diff |
| BLANK | 1A01 | 0.00 | (0.079) | | | |
| | 1B01 | 0.00 | (0.079) | | | |
| | avg | 0.00 | (0.079) | | | |
| ZERO | 1C01 | 0.00 | −0.010 | | | |
| | 1D01 | 0.00 | −0.012 | | | |
| | avg | 0.00 | −0.011 | | | |
| STANDARD A | 1E01 | 5.00 | 0.022 | 4.30 | | −14.0 |
| | 1D01 | 5.00 | 0.033 | 5.87 | | 17.4 |
| | avg | 5.00 | 0.028 | 5.08 | 21.8 | 1.7 |
| STANDARD B | 1G01 | 25.00 | 0.149 | 24.36 | | −2.8 |

| | | ASSIGNED | | Calc | CV | % |
|---|---|---|---|---|---|---|
| NAME | WELL | IU/ml | O.D. | IU/ml | % | Diff |
| STANDARD C | 1A01 | 0.00 | (0.079) | | | |
| | 1B01 | 0.00 | (0.079) | | | |
| | avg | 0.00 | (0.079) | | | |
| STANDARD D | 1C01 | 0.00 | −0.010 | | | |
| | 1D01 | 0.00 | −0.012 | | | |
| | avg | 0.00 | −0.011 | | | |
| STANDARD E | 1E01 | 5.00 | 0.022 | 4.30 | | −14.0 |
| | 1D01 | 5.00 | 0.033 | 5.87 | | 17.4 |
| | avg | 5.00 | 0.028 | 5.08 | 21.8 | 1.7 |
| STANDARD F | 1G01 | 25.00 | 0.149 | 24.36 | | −2.8 |

CONTROL ANALYSIS

| Description | Well | O.D. | Calc IU/ml | CV % | Dil Fact |
|---|---|---|---|---|---|
| Level I | 1A3 | 0.176 | 28.09 | | 3.40 |
| Mean = 15.50 | 1B03 | 0.162 | 28.82 | | 2.78 |
| 2D = 4.00 | avg | 0.169 | 27.86 | 6.3 | 3.09 |
| Level II | 1C3 | 0.343 | 61.75 | | −0.32 |
| Mean = 84.93 | 1D03 | 0.340 | 61.11 | | −0.38 |
| 2D = 10.00 | avg | 0.342 | 61.43 | 0.7 | −0.35 |
| Level III | 1E03 | 0.830 | 198.50 | | −1.96 |
| Mean = 247.50 | 1F03 | 0.946 | 243.83 | | −0.15 |
| 2D = 25.00 | avg | 0.888 | 221.17 | 14.5 | −1.05 |

LIST OF TESTS

| SAMPLE | | | | | | Sample |
|---|---|---|---|---|---|---|
| ID | Well | O.D. | Calc IU/ml | CV % | Dil Fact | IU/ml |
| 71881 | 1G03 | 0.003 | 1.71 | | 1 | 1.71 |
| | 1H03 | 0.009 | 2.51 | | 1 | 2.51 |
| | avg | 0.006 | 2.11 | 26.8 | 1 | 2.11 |
| 83878 | 1H03 | 0.007 | 2.24 | | 1 | 2.24 |
| | 1G03 | 0.003 | 1.71 | | 1 | 1.71 |
| | avg | 0.005 | 1.97 | 19.0 | 1 | 1.97 |
| 71882 | 1F03 | 0.051 | 8.52 | | 1 | 8.52 |
| | 1E03 | 0.032 | 5.78 | | 1 | 5.73 |
| | avg | 0.042 | 7.12 | 27.8 | 1 | 7.12 |
| 75881 | 1D03 | 0.006 | 0.56 | | 1 | 0.56 |
| | 1C03 | 0.003 | 1.71 | | 1 | 1.71 |
| | avg | 0.002 | 1.14 | 71.2 | 1 | 1.14 |
| 73148 | 1B03 | 0.001 | 1.45 | | 1 | 1.45 |

Results of Dengue IgM and IgG Negative-IgE

| | Laboratory | | | |
|---|---|---|---|---|
| | Identification | Dengue IgM | Dengue IgG | IgE |
| Number | Number | UM ELISA | UM ELISA | IU/ML |
| 1 | C-003 | No Reactive | No Reactive | 446.00> |
| 2 | C-024 | No Reactive | No Reactive | 49.29 |
| 3 | C-054 | No Reactive | No Reactive | 110.50 |
| 4 | C-066 | No Reactive | No Reactive | 446.00> |
| 5 | C-069 | No Reactive | No Reactive | 446.00> |
| 6 | C-073 | No Reactive | No Reactive | 163.22 |
| 7 | C-078 | No Reactive | No Reactive | 87.42 |
| 8 | R-106 | No Reactive | No Reactive | 446.00> |
| 9 | R-117 | No Reactive | No Reactive | 446.00> |
| 10 | R-119 | No Reactive | No Reactive | 75.550 |
| 11 | R-130 | No Reactive | No Reactive | 80.33 |
| 12 | L-148 | No Reactive | No Reactive | 57.83 |
| 13 | L-151 | No Reactive | No Reactive | 63.87 |
| 14 | L-161 | No Reactive | No Reactive | 80.35 |
| 15 | L-164 | No Reactive | No Reactive | 446.00> |
| 16 | L-172 | No Reactive | No Reactive | 195.69 |
| 17 | L-194 | No Reactive | No Reactive | 51.460 |
| 18 | L-201 | No Reactive | No Reactive | 145.60 |
| 19 | L-206 | No Reactive | No Reactive | 87.42 |
| 20 | SL-272 | No Reactive | No Reactive | 171.46 |
| 21 | SL-284 | No Reactive | No Reactive | 90.38 |
| 22 | SL-289 | No Reactive | No Reactive | 294.51 |

In summary, these preliminary findings indicate that elevated IgE levels are 1) evident in older adults 2) associated with mental impairment 3) may be sensitive prognostic marker of cognitive impairment. Thus we are claiming that IgE could be used as a diagnostic tool for cognitive impairment. I am including laboratory report to demonstrate the veracity of the analyzed data.

A diagnostic assay for several diseases based on IgE antibodies. IgE antibodies will be detected in Alzheimer Disease, Multiple Sclerosis, Parkinson disease, Down Syndrome, severe trauma of the central or peripheral neural system. These antibodies, may permit the early detection of CNS compromise. The IgE elevations will correlated with the severity of the disease and thus may be used as a marker of the disease and probably treatment responses.

Similarly, IgE elevations will be detected during Malignancies and will be more severely increased as the disease progresses, as well as with the size of the tumor. I also hypothesize that IgE levels will rise with the appearance of metastasis. IgE levels will decline after successful treatment and, thus may be useful as both a prognostic marker and tool for treatment follow-up. Use of IgE as a diagnostic tool may also exist for diseases caused by bioterrorism

Specific Ige Background Information that Supports the Usefulness of an IgE Diagnostic and Treatment

IgE and Neurodegenerative Disorders

Although the role of IgE in neuropathogenesis has not yet been identified, microglial cells, which are deeply integrated in the physiopatology of neurodegenerative disease, have IgE receptors that are used in microglial-T cell communication. Moreover, IgE, which triggers histamine, serotonin and cytokine (IL1, IL6, TNF) release by mast cells is the most powerful amplifier of the immune response, and has been reported to be elevated during CNS compromise. Elevation of IgE and its receptors has been described in Down's syndrome, Multiple Sclerosis, Parkinson's disease and AIDS dementia. Additionally, brain IgE sensitization in animal models has been demonstrated to increase adrenal cortisol secretion via histamine release and induce neuronal apoptosis. CD23, the low affinity receptor of IgE, is present in platelets, astroglial and microglial cells and highly expressed in patients with Parkinson's Disease (16) and HIV dementia (10), but is low or not detectable in healthy brain tissue. Additionally, CD23 antigen is co-localized with iNOS and nitrotyrosine in the areas of the brain compromised, suggesting that it participates in iNOS activation of astrocytes and CNS inflammation (10). These findings suggest a critical relationship between IgE and neuro-inflammatory reactions, constituting the scientific basis for this proposal

Alzheimer Disease

Some of the features of neurodegenerative physiopathology suggest a critical role for IgE antibodies. First, Alzeimer Disease (AD) involves accumulation of beta amyeloid, and antibodies could prevent A beta from aggregating into fibrils. Secondly, antibodies can accelerate clearance of A β by stimulating its removal by microglial cells. Rationale for an IgE role in AD, is supported by our preliminary data demonstrating that IgE levels increase with age, and are associated with cognitive decline. Together with IgE affects on neurotransmitter release and oxidative stress, these data support a plausible role for IgE in the pathology of dementia.

A more refined understanding of the physiopathology of Alzheimer dementia is clearly needed. Identifying and targeting components of the IgE signaling pathway could form the basis for new diagnostic and pharmacological approaches for the chemoprevention and treatment of CNS associated disease.

Rationale for an IgE Mechanism in Ad:

Elevated IgE and/or IgE receptors are evident in CNS diseases that involve inflammation, apoptosis, demyelinization and oxidative stress, all features of AD. IgE and Plaque Formation: IgM, IgG and microgial response are important in accelerating clearance of A p amyeloid and thus, in AD physiopathology (27). IgE's action in AD processes, however, has not yet been identified although, IL1, IL6 and PgE2 that have been suggested to modulate A β precursor protein synthesis are all modulated by IgE. Of interest, both microglial and astrocytes have IgE receptors (Chabot, Dugas).

Numerous integral membrane proteins are released from the cell surface through a post-translational proteolytic cleavage event and include the Alzheimer's amyloid precursor protein (APP), pro-TNF-α, and CD23, the low affinity receptor of IgE. Of interest, the secretase that cleaves and releases APP from the plasma membrane also releases CD23 from the cells (33).

IgE and Interleukin Production:

Considerable attention has been given to the potential contribution of pro-inflammatory cytokines in the neurodegenerative changes observed in AD. This interest is based on (a) overexpression of ILs in AD plaques; (b) IL overproduction by microglia and consequently expression in pathogenesis;^{23,22,28} and (c) genetic polymorphisms in promoter regions of ILs that have been associated with an increased risk of developing AD. ^{14,25} Similar to the age-dependent histamine findings, IL-6 increases with age, is significantly elevated in AD, and IL-6 gene variation has been associated with increased risk of AD.

Studies indicate that prolonged signaling induced by IgE may enhance cytokine secretion. Kalesnikoff and collaborators showed that IgE alone stimulates cytokine secretion by mast cells. In further support of this hypothesis, Oliveira& Lukacs demonstrated that TNFα production by mast cells was significantly higher after IgE stimulation. Similar findings have been reported with IL-6 and IL1 production (Gibbs et al 2001). IgE has been shown to down-regulate the response by inducing the expression of IL1-ra, a blocker of IL1 production. IL1-ra is produced by neuronal cells, highly expressed in brain tissue and associated with senile plaques in patients with AD (46).

IgE may by itself, thus, have the potential to increase oxidative stress, IL-6, IL-1 and TNF production, as well as promote the release of histamine by mast cells, and enhance an HPA response inducing neuronal apoptosis (25). Neuronal damage associated with microglia activation and overexpression of IL1 and IL6 provides feedback to continue the amplification of the immune response.

Activated microglia expressing IL-1 and elevated IL-6, in close proximity to amyloid plaques have been reported, as well as, an IL-1 and IL-6 induced increase in the synthesis of amyloid precursor proteins.³⁶ Of importance, IL-6 is the terminal differentiation factor that causes B cells to become plasma cells and, in the presence of IL-4, induces IgE secretion. To complete the process IgE, in turn, increases the production of IL-6. Moreover, IL1 which is elevated in AD, is well known for inducing the production of IL-6 and TH2 cytokines by mast cells, enhancing IgE production and co-expression of IgE receptors.^{30,21}

TNF-alpha elevation has been described in AD and implicated as a potent neurotoxic agent. Meda et al demonstrated that β amyeloid induces, in an age-dependent way, the production of TNF by microglia. Of interest, IgE pre-stimulation has also been shown to enhance TNF production and release by microglia cells.

IgE, Histamine and Alzheimer's Disease:

In addition to the humoral response, other elements of the immune system such as histamine have been associated with AD. Histamine, a messenger molecule in cell-to-cell communication, is produced and stored in mast cells, basophils and neurons. Approximately 60-80% of the total histamine is in the neuronal pool. Additional non-neuronal pools for histamine in the brain include, mast cells, glial, and vascular endothelial cells. Mast cells contain IgE receptors that release histamine and serotonin when IgE and/or cytokines bind to them.12 An age-dependent increase in histamine content has been observed in areas of release (hypothalamo-infundibular, hypothalamo-hypophysial) and a decrease in areas of synthesis.

Recent data indicate that serum histamine levels are significantly higher in AD patients (10.935+/−5.692 nM) than in controls (5.533+/−2.567 nM), and correlated with alterations in neuroendocrine, cognitive, neurovascular, sleep-wakefulness functions and pulsatility index scores (decrease in blood flow, and increase in resistance).8 Of interest, histamine released by both neurons and mast cells, evokes a hypothalamic-pituitary-adrenal response. In passively IgE-sensitized dogs, activation of mast cells leads to a marked increase in activation of the HPA axis. IgE sensitization was achieved by either central or peripheral production and may explain alterations in the permeability of the blood brain barrier²⁴. These data underscore the need for an enhanced vision of IgE in the CNS physio-pathology of AD. Increased histamine release by mast cells, which is triggered by IgE, has been associated with AD. IgE not only activates release of histamine, serotonin and ILs from mast cells, Asai has shown that IgE suppresses apoptosis in mast cells. Thus, IgE-dependent enhancement of mast cell survival may explain, at least in part, the increase in/histamine during AD.

IgE and Oxidative Stress:

CD23, the low affinity receptor of IgE, has been implicated in the induction of iNOS in the immune system. I postulate that interleukin-1β and TNF-α can induce the expression of CD23 in glial cells, produce oxidative stress and induce apoptosis. Conversely, it has been shown that the engagement of the CD23 molecule also regulates the production of proinflammatory cytokines such as TNF-α and IL-6 (Arock et al., 1994; Mossalayi et al.) completing the vicious cycle. The schematic figure below illustrates the possible role of IgE in the neuropathogenesis of Alzheimer's disease.

[image]

Four major loci in chromosomes 21, 14, 1, and 12 have been linked to AD. These mutations have been postulated to result in alterations of the mechanisms processing APP, a trans-membrane precursor of -amyeloid. Studies suggest that the genetic model underlying IgE does not consist of a single major gene, but of multiple loci with more modest effects. Both approaches have identified putative quantitative trait loci on chromosomes 1, 14 and 21 (Alarcon, Soderhall et al 2001).

Another interesting feature of AD, which constitutes the rationale for initially measuring a systemic immune response, is that immune abnormalities observed in AD patients are not limited to the CNS. Togo et al. (2002) have described an increased accumulation of activated, but not well differentiated, T cells in the brain parenchyma of AD patients. Stieler et al. (2001) demonstrated that stimulated peripheral blood lymphocytes from AD patients were less able to express CD69, an early proliferation marker, than in age-matched controls. Moreover, the expression of CD69 was inversely correlated with Minimental State Examination scores. Finally, increased production and expression of some interleukins (IL1, IL6, TNF)—have been described not only in, and surrounding the senile plaques, but also at the systemic level.³ Even though evidence suggests a critical role for IgE in orchestrating the cascade of immune events that occurs in AD, the specific function and action of IgE remains unclear. Enhanced understanding of IgE action may lead to the potential development of therapeutic targets to slow and prevent AD.

Rationale for an IgE Mechanism in Parkinson Disease:

Although no articles exist, to the best of our knowledge regarding total or specific IgE in patients with Parkinson, indirect data indicate the involvement of IgE. Similar to Alzheimer Disease, histamine levels have been found to be elevated in patients with Parkinson disease but not in control subjects. Histamine concentrations were significantly increased in the putamen (to 159% of the control mean), substantia nigra pars compacta (to 201%), internal globus pallidus (to 234%) and external globus pallidus (to 200%), i.e. in areas which play a crucial role in the motor behaviour and which show typical functional alterations in PD. Of interest, after treatment and improvement patients with Parkinson exhibited a decrease in circulating histamine levels.

IgE and IgE Receptors in Multiple Sclerosis:

Multiple Sclerosis, are considered to be CD4+ T-Cell-mediated AutoImmune Diseases affecting the Central Nervous System. For some authors Multiple sclerosis is mediated by an inflammatory TH1 response. Several lines of indirect evidence suggest that IgE system of interleukins and Mast Cells could also play a role in the PathoGenesis of Multiple Sclerosis. Moreover, data from Ochi suggest that CD8+ T cells play an adjunctive role in disease induction and the clinical course of Multiple Sclerosis. Our data indicated a positive relationship between CD8 and IgE production. Similar to Alzheimer disease Mast Cells have been identified in patients with Multiple Sclerosis amongst perivascular inflammatory cells as well as free in the parenchyma, especially inside and around ‘chronic active’ plaques. Staining for IgE demonstrated moderately strong on and within MCs, and weak within some plasma cells. Thus Mast cells and IgE, could conceivably have played some role in the pathogenesis of the MS plaques (Journal of Neuroimmunology. 30(2-3):169-77, 1990). After 1990 controversial data has been published on regards to IgE and Multipe Sclerosis. While Horiuchi and collaborators described and increased frequency of mite-allergen reaction on patients with multiple sclerosis. Oro and colleagues described significantly fewer allergic symptoms, a lower number of positive allergen-specific IgE test results, and lower composite allergy indexes in patients with multiple sclerosis than control subjects. The authors suggest that the low prevalence of IgE-mediated allergic disease is associated with genetic factors that promote susceptibility to Th1-mediated inflammatory disease in human beings that thus protect them against the development of Th2-mediated disease.

Elevations of total IgE have also been detected in Down's syndrome, which has similar neuropathologic characteristics of AD (19). Sale and colleagues recently described a clinical case of Churg-Strauss vasculitis, in remission, that recurred with hyper IgE and microamyloid deposits, further suggesting a role of IgE in the amyeloid deposit mechanism.

Preliminary Studies:

Measurements of Mental Status, Immunoglobulins and Cytokines: Although the specific findings of our HIV(+) and HIV(−) groups cannot be extrapolated directly to Alzheimer patients, our results provide preliminary information regarding IgE's action in mental function. These preliminary studies revealed:

1. IgE levels were higher in the patients older than 40 yrs (387±331 IU/ml) relative to younger patients (329±341 IU/ml). FIG. 1a indicates lower IgE levels in HIV(−) less than 45 years of age (n=11; 97±145), compared to older seronegatives >45 (n=3; 198±246 IU/ml).

2. Mean IgE levels were significantly lower, however, in patients with MMSE total scores >28 (238±326 IU/ml), compared to those who scored <27 (483±336, p=0.04). Measurements of IgE levels were higher in the impaired group (<24): 73% had more than 200 IU/ml of total IgE. Univariate analyses demonstrated that patients with IgE>200 IU/ml were 6 times more likely to be impaired (MMSE score <24), as compared to those with <200 IgE IU/ml (p=0.02 FIG. 1B).

Prognostic Value of Total IgE in Mental Function:

Univariate analyses indicated that patients with IgE >200 at baseline were 3 times more likely to be impaired (score<24, p=0.01) at the 2 year visit. In summary, these preliminary findings indicate that elevated IgE levels are 1) evident in older adults 2) associated with mental impairment 3) may be a sensitive prognostic marker of cognitive impairment and 4) tend to be correlated with cytokine alterations observed in AD, supporting an important role for IgE in AD.

Malignancies

Antibodies have also found application in the diagnosis and therapy of cancer. Research related to these anti-tumor antibodies, however, has been confined to isotypes of IgG and little is known of the potential usefulness of other classes of immunoglobulins.

Allergic reactions, which are mediated by IgE antibodies, bound to its receptors on mast cells in peripheral tissues and are characterized by their immediacy and hypersensitivity. Once activated, mast cells release TNF-alpha and other mediators that attract inflammatory cells, such as eosinophils and macrophages to the tumor site. It may even be expected that eosinophils perform IgE-mediated lysis of tumor cells. In addition, Reali and colleagues have demonstrated in mice that IgE also elicited tumor-specific T-cell responses. Similarly, tumor vaccination experiments showed that irradiated tumor cells (IgE loaded by biotin-avidin bridging) conferred protective immunity at doses 100-fold lower than the corresponding control cells without IgE (Cancer Res 2001 Jul. 15; 61(14):5517-22). To further support the important role of IgE on malignancies, our data, along with others, demonstrated a positive correlation between natural killer cells and IgE levels. With those on the highest percentile of IgE exhibiting high NK activities (International Archives of Allergy & Immunology. 112(4):331-5, 1997) These properties also indicated human IgE responses against different cancers, that may be used for diagnosis, prognosis and vaccines: These results suggest that tumor-specific IgE antibodies exist and may be exploited for diagnosis, prognosis and immunotherapy of cancer

IgE and Bioterrorrism

Similar to infectious diseases elevations of IgE are due to the disease and not to allergies. These are anthrax, smallpox, plague, botulinum toxin, tularemia, and viral hemorrhagic fever. herpes simplex virus (HSV), varicella-zoster virus (VZV), and Bacillus anthracis (anthrax). Anthrax remains a real threat. In its spore form, it is highly infectious with widespread dissemination. The initial symptoms are similar to those of influenza, and the early stage of inhalational anthrax may not be recognized. Prevention and adequate response to anthrax infection requires early detection and the development of a vaccine. The Clinical course varies according to the point of bacillus entrance.

Cutaneous:

The mean incubation period for cutaneous anthrax has been estimated as 5 days (range: 1-10 days). The patient can present with small, erythematous, tingling sensation and/or pruritic papule that later develops into a small vesicle. About 20% of untreated cases of cutaneous anthrax will result in death.

Respiratory Characteristics:

The median age of the 10 patients with inhalational anthrax was 56 years (range: 43-73 years); seven were men. The incubation period from the time of exposure to onset of symptoms when known (seven) was 7 days (range: 5-11 days). The initial illness in these patients was characterized by fever (nine) and/or sweats/chills (six) Severe fatigue or malaise was present in eight and minimal or nonproductive cough in nine, including one with blood-tinged sputum. Eight patients reported chest discomfort or pleuritic pain. Abdominal pain or nausea or vomiting occurred in five, and five reported chest heaviness. Other symptoms included shortness of breath (seven), headache (five), myalgias (four), and sore throat (two).

Inhalation Anthrax is Usually Fatal.

On initial presentation, total WBC count was normal or slightly elevated (7.5-13.3×103/cumm); however, elevation in the percentage of neutrophils or band forms was frequently noted. None of the patients had a low WBC count or lymphocytosis when initially evaluated. Chest radiograph was abnormal in all patients, but in two an initial reading was interpreted as within normal limits. Mediastinal changes including mediastinal widening, paratracheal fullness, hilar fullness, and mediastinal lymphadenopathy were noted in all eight patients who had CT scans. Mediastinal widening may be subtle, and careful review of the chest radiograph by a radiologist may be necessary. Pleural effusions were present in seven patients and were a feature of the two patients who did not have mediastinal changes on chest radiograph or did not have a CT scan. Pleural effusions often were large and hemorrhagic, reaccumulated, and required repeated thoracentesis or chest tubes. Pulmonary infiltrates were observed in four patients and were multilobar in three. Blood cultures grew B. anthracis in seven patients and in all who had not received antimicrobials. Diagnosis in the patients with negative cultures was confirmed by bronchial or pleural biopsy and specific IHC staining, by PCR of material from a sterile site, or by a fourfold rise in IgG to the protective antigen.

Gastrointestinal:

The intestinal disease form of anthrax may follow the consumption of contaminated meat and is characterized by an acute inflammation of the intestinal tract. Initial signs of nausea, loss of appetite, vomiting, fever are followed by abdominal pain, vomiting of blood, and severe diarrhea. Intestinal anthrax results in death in 25% to 60% of cases.

Based on the clinical course I am going to discuss why IgE may be useful:

1. It needs to be highlighted first that the incubation period is as short as 1-5 days, thus to attain early diagnosis only IgM and IgE can be used.

2. The clinical dermatological cases involving pruritus, which are mostly associated with IgE.

3. Sytemic allergic reactions are characterized mainly by their severe inflammatory component, shortness of breath and effusions. This clinical course is similar to the anthrax cases described above.

4. In approximately 20% of the cases, blood cultures are false negatives thus resulting in an unacceptable low specificity

Form the immunological point of view, systemic pro-inflammatory cytokine release has been previously implicated as a major death-causing factor in anthrax. Balb/c mice have also responded with higher levels of IL-6. Of interest IL-6 is associated with the induction of IgE.

Coronavirus Associated with Severe Acute Respiratory Syndrome (SARS)

SARS is a recently recognized infectious disease caused by the SARS-associated coronavirus (SARS-CoV), a new member in the family Coronaviridae. The virus produces severe respiratory failure characterized by acute-phase diffuse alveolar damage (DAD), airspace edema, and bronchiolar fibrin. Cases of more than 10 days' duration exhibited organizing-phase DAD, type 11 pneumocyte hyperplasia, squamous metaplasia, multinucleated giant cells, and acute bronchopneumonia. It is a disease associated with severe morbidity and mortality.

Since November 2002, the Corona-virus has spread from South China to almost 30 other countries, where about 8500 infected individuals have been registered; approximately 800 people have already died from the disease (9.5%). It presents with non-specific signs and symptoms and because no definitive laboratory test is readily available, it poses a great public health risk. Its very short incubation period makes strategic health responses a very difficult task. Based on 42 of the 128 cases with a single known contact with a SARS case, the mean incubation period was 5 days (range 2 to 10 days).

Early recognition and isolation of a possible source are an essential part of the critical response to an outbreak. To achieve this goal an assay capable of detecting the coronavirus during the first week is needed. To understand the time-course of viraemia and antibody responses to SARS, Chen and colleagues studied 376 blood samples from 135 SARS patients at various stages of the illness. The results showed that IgM antibodies decreased and became undetectable 11 weeks into the recovery phase. IgG antibodies, however, remained detectable for a period beyond 11 weeks. SARS-CoV viraemia mainly appeared 1 week after the onset of illness and then decreased over a period of 1 month, becoming undetectable in the blood samples of the convalescent patients. Only at the peak of viraemia was, viral RNA detectable in 75% of blood samples from SARS patients diagnosed 1 or 2 weeks before the test.

Based on these results IgM and IgG were evaluated in detection assays against the N protein: The IgM positive critical value of 0.233 and IgG of 0.239 were selected as the threshold value for positive results that equals the product of 2.1 and the mean IgM and IgG levels of 200 healthy blood donors. In 13 patients with SARS, the antibody responses to N antigen were not detectable in the first week after the onset of symptoms. The IgM and IgG seroprotection rates were 83.3% and 66.7% respectively in the second week, both reaching 100% at the third week. IgM seroprotection rates were 61.5% in the second month, and 38.5% at third month. The IgG peaked one month after the onset and remained at high levels in the following 2 months.

The antibody responses suggest that humoral responses are an important component of responses to N protein of SARS is immunodominant, however, and the antibodies assayed are not adequate for detection/diagnosis. PCR technology was also evaluated, with limited results. Nasopharyngeal swab specimens were negative for the SARS-associated coronavirus by an in-house reverse transcriptase-polymerase chain reaction in all 25 children.

SARS and Immune Response

The number of total B cells of clinically diagnosed SARS was (292+/−181)×10(6)/L, significantly greater than that of healthy adults which was (200+/−65)×10(6)/L (F=6.17, P<0.05). Moreover, The number of B cells in severe SARS and mild cases were (347+/−156)×10(6)/L and (268+/−211)×10(6)/L respectively. (Chinese Journal of Tuberculosis & Respiratory Diseases. 26(10):590-3, 2003) In addition, stimulated IL-10 secreting cells increased in early SARS but declined during treatment. In contrast SAC-induced IL-12 secreting cells were deficient before, during and long after treatment. Numbers of cells induced to produce IL-6 and tumour necrosis factor-alpha by T cell or monocyte activators were higher than normal in many early SARS patients and were still increased in some during and after treatment. The described dysregulated cytokine production that occurs in SARS is compatible with a switch towards IgE production, thus further supporting our hypothesis.

All diagnostic assays are based on IgE antibodies. IgE antibodies will bind to the diverse antigens or oncogens eliciting an antigen antibody response that could be marked, The early production of IgE even with small amounts of antigen will guarantee a production of IgE by the individual affected by the proposed disease. As the immune system fails to control the disease a more dramatic IgE response will be observed.

The IgE based methodology to diagnose infectious diseases includes our previously described ELISA IgE based assay, that will now be used in other infectious diseases, a fluorescent IgE method, as well as an IgE based rapid test. All of these IgE methods are easy to employ, cost effective and could be useful worldwide.

The ELISA IgE Method:

Samples of serum or plasma are pretreated by the protein G affinity method (rProtein G Affinity Method; Isolab, Inc., Akron, Ohio). The sample is added to the resin tube and incubated for 10 minutes. A special disk is then inserted into the tube and pressed down to compress the resin bed. The supernatant is ultimately used for testing. Each well will be coated with the corresponding antigen (i.e. myelin glycoprotein, apoE, N protein of the coronavirus). After this, the plates will be incubated at room temperature with the resin-treated serum (diluted 1/50) for 30 minutes and then aspirated and washed five times with 300 μl of wash solutions per well. Horseradish peroxidase-conjugated with anti-human IgE and anti-CD23 will be added, and the mixture will be incubated for an additional 30 min at room temperature. Plates will be re-aspirated and re-washed five times with 300 μl of wash solutions per well. Subsequently, 100 μl of fresh substrate solution will be added to each well, followed by incubation at room temperature. The reaction will be stopped by adding 100 μl of stop solution (1 N H₂SO₄) to each well. The plate will be read with bichromatic absorbance at 492 with 620 as a reference marker. As the color intensity is directly related to the concentration of the anti-antigen IgE antibodies we expect to see higher absorbance among those suffering the most severe cases, compared to mild cases or those of healthy individuals. The IgE principle can also be applied in rapid diagnostic tests, thus our second proposed goal is to develop a rapid test based on IgE for the proposed diseases.

Diagnosis and Treatment of neurodegenerative diseases, such as Alzheimer's disease, multiple sclerosis, industry. Parkinson's disease and amyotrophic lateral sclerosis (ALS), represents a major challenge for the pharmaceutical. These disorders have common and unique molecular pathological characteristics, an inflammatory cascade mediated by IgE that result in serious reductions in nervous-system functionality. Key to developing novel diagnostic and efficacious therapeutics is the discovery of an specific IgE response. Mediating the physiopathology of these diseases. A diagnostic test for diseases in which an inexpesive and accurate test is not currently available. In addition, there are no markers of disease progression for the above described diseases. Such a tool could be very helpful for clinicians. Since IgE assays are inexpensive they could have widespread and global usefulness. In addition, malignancies are a leading cause of morbidity and mortality in the world, and have been declared a global emergency. Conventional techniques such as MRI and TACS are, either not sufficiently efficient and specific, or are extremely expensive and require an extended turnaround/time from the laboratory to performed biopsy studies. An IgE ELISA test for cancers, leukemias, lymphomas etc will improved medical diagnosis, reducing errors and will greatly improve public health care systems worldwide.

Bioterrorrism

Detection of antibody to SARS CoV is useful in the diagnosis of SARS; however, at the incubation and initial phases of the illness, serological and virological assays are of little value, because of late seroconversion in most patients. IgE has not been evaluated in the immune-response nor in the diagnosis of SARS epidemic. The early response of IgE, however, and the increased sensitivity of the IgE assays during other infectious diseases strongly suggest that IgE may be useful on the early diagnosis of SARS, thus helping to control the spread of the disease during future SARS outbreaks. (Pediatrics. 112(4):e261, 2003).

What are the standard diagnostic tests used by the laboratories in the diagnosis of anthrax? Presumptive identification to identify to genus level (Bacillus family of organisms) requires Gram stain and colony identification. Presumptive identification to identify to species level (B. anthracis) requires tests for motility, lysis by gamma phage, capsule production and visualization, hemolysis, wet mount and malachite green staining for spores. Confirmatory identification of B. anthracis carried out by CDC may include phage lysis, capsular staining, and direct fluorescent antibody (DFA) testing on capsule antigen and cell wall polysaccharide. The length of time needed depends in part on how fast the bacteria grow, but results are usually available 1 to 3 days after the sample is received in the laboratory.

IgE tests can be designed that are applicable to a wide spectrum of microorganisms and may be used in a clinic or far-forward deployed setting to aid in diagnosis of disease or verification of vaccination.

After carefully review the main medline sources (OVID, PUBMED, Medscape, internet) our literature search demonstrated that currently, scientific publications demonstrating total or specific IgE responses are unavailable for the following diseases (Alzheimer Disease, Parkinson, SARS, herpes virus and antrax). No articles has described not even hypothesized that IgE could be useful in the development of diagnostic assays. Moreover there is no commercial IgE diagnostic method for the following diseases (Alzheimer, Parkinson Disease, Multiple sclerosis, malignancies, lymphomas, leukemias, SARS, herpes infection, anthrax). Thus we are claiming: An IgE response in all of the above diseases. We are claiming a new group of IgE based methods, using an IgE ELISA/EIA/rapid system, to accurately diagnose these neurological diseases We are claiming the presence of an IgE response during Alzheimer disease including tau proteins, alpha-, beta-, and gamma-synuclein, A beta 40 and 42. We claim a specific IgE response against the myelin proteins such the olygodendrocyte glycoprotein and C8. We claim a specific IgE response against the dopaminergic cells in Parkinson disease. We are claiming that modifications of the IgE cascade can be used in the treatment of neurodegenerative diseases, such as Alzheimer's Disease, Parkinson, Multiple Sclerosis, Down Syndrome and HIV associated dementia. We are claiming IgE anti-dopamine antibodies in Parkinson patients. We are claiming that IgE based diagnostic assays permit the early and accurate diagnosis of cancers, malignancies, lymphomas and leukemias. We are claiming that there is a specific IgE response against the N Protein of the coronavirus. We are claiming that there is a specific IgE response against anthrax antigens and toxins that will permit the early diagnosis and development of protective vaccines or treatments. We claim that IgE levels can be used in diagnostic tests and that the use of IgE ELISA or EIA can distinguish between mild, moderate and advanced stages of the disease. As the color intensity is directly related to the concentration of the anti-antigen IgE antibodies we expect to see higher absorbance among those suffering more severe forms of the disease, compared to acute or mild forms of the clinical picture.

There is a critical need for diagnostic and therapeutic strategies to slow and/or prevent neurodegenerative diseases. Manipulation of the IgE system has the potential to introduce vital information and a new dynamic into the diagnosis of neurological diseases. Over the past several years we have conducted numerous studies of antioxidants, interleukins and IgE, in an effort to strengthen our understanding of the dynamic relationships between these three elements in health and disease. Our pioneering studies first described the role of IgE in HIV/AIDS and its potential prognostic value. This method could be easily applied to diagnose both infectious diseases as well as any disease that may not be initially controlled by a TH1 response. We have previously demonstrated that IgE increases with age and is associated with cognitive decline. In the near future we plan to explore which antigens are involved in generating an IgE related response during these diseases

### REFERENCES

1. Alarcon M and Cantor R M. Quantitative trait loci mapping of serum IgE in an isolated Hutterite population. Gen Epi 2001; 21(S1):S224-9.
2. Alexopoulos G S et al. Cornell Scale for Depression in dementia. Bio Psych 1988; 23; 271-284.
3. Akiyama H et al. Neurobio Aging 2000; 21(3):383-421.
4. Arock M et al. Involvement of Fc epsilon R11/CD23 and L-arginine dependent pathway in IgE-mediated activation of human eosinophils. Biochem & Biophys Res Comms 1994; 203(1):265-71.
5. Asai K et al. Regulation of mast cell survival by IgE. Immunity 2001; 14(6):791-800.
6. Bard F C et al. Peripherally administered Abs against amyloid beta-peptide enter the central nervous system and reduce pathology in a mouse model of Alzheimer disease. Nat Med 2000; 6:916-19.
7. Cacabelos R. Brain histamine: psychoneuroimmune regulator in physiological and pathological conditions. J Nutri Sci & Vitamin 1992; 573-6.
8. Cacabelos R et al. Brain histamine in Alzheimer's disease. Methods & Findings in Exp & Clin Pharm 1989; 11(5):353-60.
9. Chabot S et al. Cytokine production consequent to T cell—microglia interaction: the PMA/IFN gamma-treated U937 cells display similarities to human microglia. J Neurosc Methods 2001; 105(2): 111-20.
10. Dugas N et al. Role of CD23 in astrocytes inflammatory reaction during HIV-1 related encephalitis. Cytokine 2001; 15(2):96-107.
11. Esiri M M et al. Prevalence of Alzheimer plaques in AIDS. J Neurol, Neurosurg & Psych 1998; 65(1):29-33.
12. Fernandez-Novoa L and Cacabelos R. Histamine function in brain disorders. Behav Br Res 2001; 124(2):213-33.
13. Folstein M F et al. “Mini-mental state”: A practical method for grading the cognitive state of patients for the clinician. J Psych Res 1975; 12(3):189-98.
14. Franceschi C et al. Neuroinflammation and the genetics of Alzheimer's disease: the search for a proinflammatory phenotype. Ageing 2001; 13:163-170.
15. Gibbs B F et al. Human skin mast cells rapidly release preformed and newly generated TNF-alpha and IL-8 following stimulation with anti-IgE and other secretagogues. Exp Derm 2001; 10(5):312-20.
16. Hunot S et al. FcepsilonRII/CD23 is expressed in Parkinson's disease and induces, in vitro, production of nitric oxide and tumor necrosis factor-alpha in glial cells. J Neurosci 1999; 19(9):3440-7.
17. Ibrahim M Z et al. The mast cells of the multiple sclerosis brain. J Neuroimm 1996; 70(2):131-8.
18. Johannsen P and Mai J. Alzheimer-type dementia and Down syndrome. Ugeskr Laeger 1995; 157(8): 1021-4.
19. Kadowaki J I et al. Serum IgE concentration in Down's syndrome. J Mental Defic Res 1976; 20(2):105-7.
20. Kalesnikoff J et al. IgE stimulates signaling pathways in mast cells that lead to cytokine production and cell survival. Immunity 2001; 14(6):801-11.
21. Kendall J C et al. Promotion of mouse fibroblast proliferation by IgE-dependent activation of mouse mast cells: role for mast cell tumor necrosis factor-alpha and transforming growth factor-beta 1. J All & Clin Immunol 1997; 99(1 Pt 1):113-23.
22. Li Y et al. Neuronal-glial interactions mediated by interleukin-1 enhance neuronal acetylcholinesterase activity and mRNA expression. J Neurosci 2000; 20: 149-55.
23. Loewenstein 1989
24. Lukiw W J and Bazan N G. Neuroinflammatory signaling upregulation in Alzheimer's disease. Neurochem Res 2000; 25:1173-84.
25. Matsumoto I et al. Brain mast cells act as an immune gate to the hypothalamic-pituitary-adrenal axis in dogs. J Exp Med 2001; 194(1):71-8.
26. McCusker S M et al. Association between polymorphism in regulatory region of gene encoding tumour necrosis factor alpha and risk of Alzheimer's disease and vascular dementia: a case-control study. Lancet 2001; 357:436-39.
27. McRae A et al. Microglial in neurodegenerative disorders: emphasis on Alzheimer's disease. Gerontology 1997; 43(1-2):95-108.
28. Meda L. et al. Modulation of proinflammatory cytokine release from human polymorphonuclear leukocytes by gamma interferon. Cellular 1 mm. 199; 4157(2):448-61.
29. Mossalayi M D et al. CD23/Fc epsilon RII: signaling and clinical implication. Intl Revs Immunol 1997; 16(1-2):129-46.
30. Mulder C et al. Genetic and biochemical markers for Alzheimer's disease: recent developments. Ann Clin Biochem 2000; 37(Pt 5):593-607.
31. Oliveira S H and Lukacs N W. Stem cell factor and IgE-stimulated murine mast cells produce chemokines (CCL2, CCL17, CCL22) and express chemokine receptors. Inflamm Res 2001; 50(3):168-74.
32. Parker L C et al. IL-1 beta signalling in glial cells in wildtype and IL-1R1 deficient mice. Brit J Pharm 2002; 136(2):312-20.
33. Parkin E T et al. Structure-activity relationship of hydroxamate-based inhibitors on the secretases that cleave the amyloid precursor protein, angiotensin converting enzyme, CD23, and pro-tumor necrosis factor-alpha. Biochem 2002; 41(15):4972-81.
34. Payet-Jamroz M et al. Suppression of IgE responses in CD23-transgenic animals is due to expression of CD23 on nonlymphoid cells. J Immunol 2001; 166(8):4863-9.
35. Peruyera and Sevush
36. Pfeiffer E. A short portable mental status questionnaire for the assessment of organic brain deficit in elderly patients. J Amer Ger Soc 1975; 23(10):433-41.
37. Reisberg B et al. Behavioral pathology in Alzheimer's disease (BEHAVE-AD) rating scale. Intl Psychoger 1996; 8(S3):301-8.
38. Rogers J. An IL-1 alpha susceptibility polymorphism in Alzheimer's disease: New fuel for the inflammation hypothesis. Neurol 2000; 55(4):464-5.
39. Sale S and Patterson R. Recurrent Churg-Strauss vasculitis. With exophthalmos, hearing loss, nasal obstruction, amyloid deposits, hyperimmunoglobulinemia E, and circulating immune complexes. Arch Int Med 1981; 141(10):1363-5.
40. Sevush S and Leve N. Denial of memory deficit in Alzheimer's disease. Amer J Psy 1993; 150(5):748-51.
41. Smits H A et al. Role of macrophage activation in the pathogenesis of Alzheimer's disease and human immunodeficiency virus type 1-associated dementia. Eur J Clin Invest 2000; 30(6):526-35.
42. Soderhall C et al. Linkage and association to candidate regions in Swedish atopic dermatitis families. Hum Gens 2001; 109(2):129-35.
43. Stieler J T et al. Impairment of mitogenic activation of peripheral blood lymphocytes in Alzheimer's disease. Neuro Report 2001; 12(18):3969-72.
44. Toms R et al. Identification of IgE-positive cells and mast cells in frozen sections of multiple sclerosis brains. J of Neuroimmunol 1990; 30(2-3):169-77.
45. Togo T et al. Occurrence of T cells in the brain of Alzheimer's disease and other neurological diseases. J Neuroimmunol 2002; 124(1-2):83-92.
46. Yasuhara O et al. Expression of interleukin-1 receptor antagonist protein in post-mortem human brain tissues of Alzheimer's disease and control cases. Acta Neuropathologica 1997; 93(4):414-20.
   MMSE Minimental State Examination

If the patient have between <24 in the total score the patient has severe cognitive impairment. If the patient has 24-26 there is moderate cognitive impairment. Patients with 27-28 minimun. Patients with total scores between 29-30 have normal cognitive status.

“Infant patient” is defined as a baby in the age range between zero and 6 months of age; “child patient” is defined as a child in the age range 7 months to 2 years.

1. A method for detecting HIV in a baby patient or a child patient that might be infected with HIV, comprising:
obtaining a sample from the patient;
contacting said sample with IgE that binds immunologically to either 120 or hp 160, and also at least one antigen selected from the group consisting of: gp 41, pol 66, gag 55, p32, env 18 and p24 to provide an antibody-antigen complex;
detecting said antibody-antigen complex;
whereby the presence of said antibody-antigen complex in said sample indicates said patient has HIV.

2. The method according to claim 1, wherein said step detecting said antibody-antigen complex is performed by either ELISA, Western Blot, or Rapid Test.

3. The method according to claim 1, wherein said step detecting said antibody-antigen complex is performed by ELISA, wherein above about O.D. of 0.5 this indicates to 99% probability level that said patient has HIV, and between zero and 0.26 O.D. this indicates that said patient does not have HIV, and between 0.27 and 0.5 O.D. this indicates that said patient is sero-converting.

4. A method for distinguishing between classic dengue and dengue hemorrhagic fever in a patient that is believed to have either the classic of hemorrhagic version of dengue, comprising:
obtaining a sample from the patient; and
measuring the levels of IgE in said sample,
wherein if the IgE level is normal in the range between about 1 and 100 IU/ml the patient is classified as probably not having either the classic of hemorrhagic forms of dengue fever,
wherein if the IgE level is in the range between about 110 IU/ml and 449 IU/ml tis indicates that the patient has classic dengue fever, and
wherein if the IgE level is in the range between about 450 IU/ml and 1000 IU/ml this indicates that the patient has hemorrhagic dengue fever.

5. A method of using IgE as a diagnostic tool for detecting cognitive impairment in a patient who is more than 40 years old, comprising:
obtaining a sample from the patient;
measuring the levels of IgE in said sample,
wherein if the IgE level is normal in the range between about 0 and 99 IU/ml the patient is classified as having zero cognitive impairment,
wherein if the IgE level is in the range between about 100 and 239 IU/ml the patient is classified as having zero cognitive impairment,
wherein if the IgE level is in the range between about 240 IU/ml and 480 IU/ml this indicates that the patient has moderate cognitive impairment, and
wherein if the IgE level is above 480 IU/ml this indicates that the patient has severe cognitive impairment.
